(19) United States
(12) Patent Application Publication
(10) Pub. No.: US 2020/0034707 A1
(43) Pub. Date: Jan. 30, 2020

(54) Neural Network Encoders and Decoders for Physician Practice Optimization

(71) Applicant: drchrono Inc., Sunnyvale, CA (US)

(72) Inventors: Daniel Kivatinos, Mountain View, CA (US); Michael Nusimow, Mountain View, CA (US); Martin Borgt, Sunnyvale, CA (US); Soham Waychal, Sunnyvale, CA (US)

(21) Appl. No.: 16/457,769

(22) Filed: Jun. 28, 2019

Related U.S. Application Data
(60) Provisional application No. 62/703,876, filed on Jul. 27, 2018.

(51) Int. Cl. G06N 3/08; G06N 3/04; G16H 40/20

(52) U.S. Cl. CPC G06N 3/08; G16H 40/20; G06N 3/0481

(57) ABSTRACT

A machine learning system may be trained to predict codes for a physician code schedule. The machine learning system may use one or more encoders that encode codes, code schedules, and claims into separate vector spaces, where the vector spaces relate similar entities. The encoded codes, code schedules, and claims may be decoded by a decoder to predict a code to add to the code schedule. The encoders and the decoder may be machine learning models that are trained using ground-truth training examples.

### CROSS-REFERENCE TO RELATED APPLICATIONS

This application claims the benefit of U.S. Provisional Patent Application No. 62/703,876, filed Jul. 27, 2018, which is hereby incorporated by reference in its entirety.

### FIELD OF THE INVENTION

The present invention relates to using machine learning to automatically suggest additional codes for a physician's fee schedule, also referred to herein as a physician code schedule.

### BACKGROUND

When physicians bill for a treatment, such as a procedure or office visit, they record the treatments using standardized codes. These standardized codes are submitted to the payer, such as an insurance company, Medicaid, or Medicare. For example, standardized codes may be used for procedures like examining the patient's knee or setting a broken arm. Codes are sometimes numerical or alphanumeric. Some code systems include CPT, ICD-9, ICD-10, SNOMED, LOINC, RxNorm, HCPCS, and others. Some code systems are more specialized for diagnosis, while others are more specialized for procedures and payment. Some code systems have as many as tens of thousands of codes.

Partly due to the overwhelming number of codes, physicians maintain fee schedules, which are lists of the codes commonly used in their practice. This allows physicians to see what codes they commonly use for particular procedures. For example, a procedure as simple as setting a broken ankle may involve multiple codes, rather than just one, and the physician refers to his or her fee schedule to determine what codes to record when submitting the bill to the payer.

However, a problem in the art is that physicians may not know the best codes to include in their fee schedule and may not know all of the relevant codes to include in their fee schedule. This is an acute problem because of the large number of codes in many coding systems, and the opaqueness of payer policies that result in physicians not knowing which codes will be paid. One result of the problem is that new physicians may have difficulty setting up their own practices because they do not know how to correctly do billing, unless they have spent several years in training at an existing practice. Moreover, many physicians may not be optimizing their billings simply because they do not know the correct codes, leading some physicians to collect more payment for the same work for administrative reasons rather than quality of work.

### SUMMARY OF THE INVENTION

Some embodiments relate to a machine learning system for predicting billing codes for a physician fee schedule. The machine learning system can be used to suggest additional billing codes that may be added to a physician's existing fee schedule. The predictions may be made based on the existing billing codes in the physician's fee schedule and also the physician's recently used billing codes in their recent billing claims.

In one embodiment, a billing code encoder is provided that encodes billing codes into a first vector representation. In one embodiment, a fee schedule encoder is provided that encodes fee schedules into a second vector representation. In one embodiment, a billing claims encoder is provided that encodes sets of recent billing claims into a third vector representation. Each vector representation relates similar entities in a vector space by locating them closer together and causes dissimilar entities to be farther apart in the vector space.

In one embodiment, a physician fee schedule is provided and a set of recent billing claims of the physician are provided. The individual billing codes in the fee schedule and set of recent billing claims are encoded using the billing code encoder. The fee schedule is then encoded by the fee schedule encoder, and the recent set of billing claims are encoded by the billing claims encoder. The encoded fee schedule and encoded set of recent billing claims may be input to a decoder to predict a billing code to add to the fee schedule.

In one embodiment, the encoders and the decoder are machine learning models that are trained using ground-truth training examples.

### BRIEF DESCRIPTION OF THE DRAWINGS

FIG. 1 illustrates an exemplary machine learning system that may be used in some embodiments.

FIG. 2 illustrates an exemplary method of training the machine learning system.

FIG. 3 illustrate an exemplary method for using the machine learning system to output one or more predicted billing codes to add to a physician fee schedule.

FIG. 4A illustrates an exemplary method for training a billing code encoder.

FIG. 4B illustrates an exemplary method for encoding a billing code.

FIG. 5A illustrates an exemplary method for training a fee schedule encoder.

FIG. 5B illustrates an exemplary method for encoding a fee schedule.

FIG. 6A illustrates an exemplary method for training a billing claims encoder.

FIG. 6B illustrates an exemplary method for encoding a set of billing claims.

FIG. 7A illustrates an exemplary method of training a decoder.

FIG. 7B illustrates an exemplary method of outputting predicted billing codes from the decoder.

FIG. 8 illustrates an exemplary skip-gram neural network.

FIG. 9 illustrates an exemplary long short-term memory (LSTM) neural network.

### DETAILED DESCRIPTION

In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially. Also, the steps of the exemplary methods may be performed in a network environment in which some steps are performed by different computers in the networked environment.

Some embodiments relate to using one or more machine learning models to predict additional billing codes that could be added to a physician's existing fee schedule. The prediction may be made based on the physician's existing fee schedule as well as the recent billing claims made by the physician. The machine learning models may be trained based on the fee schedules and billing claims of other physicians, such as physicians who practice in a similar field or see similar patients. In this patent, the terms “physician code schedule” and “code schedule” are defined to mean a physician fee schedule.

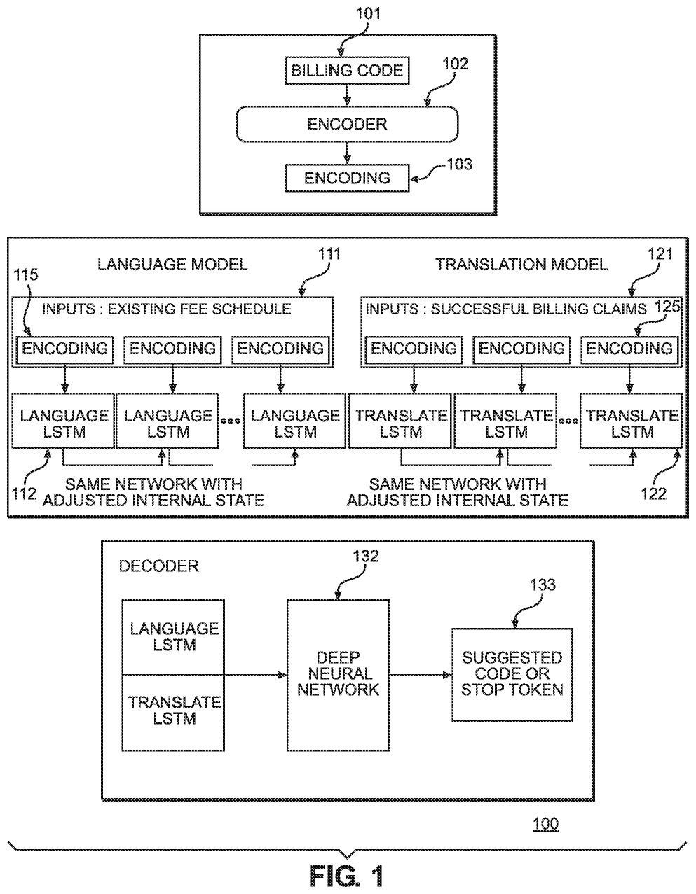

FIG. 1 illustrates an exemplary machine learning system 100 for predicting additional billing codes to add to a physician's fee schedule by using multiple machine learning models. System 100 may be embodied on a single computer system or on multiple computer systems. Encodings may be used to encode, or translate, raw text into representations that encode more information than the raw text, so that machine learning models have more information to train on. Raw text alone often fails to capture relationships with other similar concepts. Therefore, encoded representations are used herein to map concepts into a vector space. The encoded representations relate similar concepts so that similar concepts are located closer together in the vector space and unrelated concepts are located farther from each other in the vector space. This is accomplished by training a machine learning model to minimize the vector distance between similar concepts and maximize the vector distance between dissimilar concepts. Vector distance may be measured by cosine similarity, dot product, or other distance metrics.

In some embodiments, billing codes 101 are mapped into a vector representation, or encoding. In raw format, billing codes 101 may simply be numeric or alphanumeric, and therefore the raw format does not capture relationships between billing codes 101, such as the fact that billing codes 28630 and 28515 may be for related procedures that are often performed together.

A billing code encoder 102 may accept one or more billing codes 101 as input, in their raw format, and output a vector representation for each of the billing codes 101, where the vector representation relates similar billing codes. The vector representations comprise the encoded billing codes 103. The billing code encoder 102 may be implemented with a neural network. An encoder comprising a neural network may be referred to herein as a neural network encoder. In some embodiments, the encoding used for billing codes is word embedding. Some forms of word embedding include skip-gram, continuous bag of words, and Word2Vec. Billing codes may have attached information such as price, allowed price by the payer, and so forth, in addition to the treatment or procedure code itself.

A physician's fee schedule 111 is made up of one or more billing codes, which are the common billing codes used by the physician. For use in machine learning, it is desirable to encode the physician fee schedule 111 to relate similar physician fee schedules. Moreover, it is desirable to use the encoded billing code representations so that the physician fee schedule may capture information from the contextual relationships between billing codes.

A physician fee schedule 111 may initially be provided as a set of billing codes in raw format. These billing codes may be encoded by billing code encoder 102 to create encoded billing codes 115 that comprise the fee schedule 111. Fee schedules are of variable length because physicians may have varying numbers of billing codes that they commonly use. Therefore, an encoder suitable for variable length input is desirable.

Fee schedule encoder 112 may accept as input a fee schedule 111 comprising one or more encoded billing codes 115 and output an encoding of the fee schedule. The encoded fee schedule may comprise a fixed length vector representation of the variable length fee schedule 111, and the vector representation may relate similar fee schedules in vector space. In some embodiments, the fee schedule encoder 112 is a neural network. In some embodiments, the fee schedule encoder 112 is a recurrent neural network (RNN). In some embodiments, the fee schedule encoder 112 is a long short-term memory (LSTM) neural network, which is one type of RNN.

A physician's recent billing claims 121 may also be used in system 100. Each billing claim in recent billing claims 121 comprises the set of one or more billing codes submitted by the physician in one claim, which may correspond to a single visit by a patient to the physician's office. For example, if a patient received a general examination, an examination of their ankle, and setting of a broken ankle during a visit, then billing codes for each of these procedures may be included in a single billing claim. The system may select the n most recent billing claims 121 for a fixed or configurable value of n. In some embodiments, n is 5, 10, 15, 20, 25, 50, 100, or more than 100. In some embodiments, the most recent billing claims 121 are chosen by time frame, such as billing claims within the last day, last week, or last month.

The physician's most recent billing claims 121 may initially be provided as billing codes in raw text format. However, the billing codes may be input to the billing code encoder 102 to create encoded billing codes 125 of the recent billing claims 121. The billing claims encoder 122 then receives as input the recent billing claims 121 and outputs encoded recent billing claims. The encoded recent billing claims may comprise vector representations that relate similar sets of recent billing claims in vector space. The encoded recent billing claims may be a fixed length vector representation. In some embodiments, the billing claims encoder 122 is a neural network. In some embodiments, the billing claims encoder 122 is an RNN. In some embodiments, the billing claims encoder 122 is an LSTM neural network.

The encoded fee schedule and encoded set of recent billing claims may be combined, such as by concatenating the two vectors into a single vector. The vector may be input to a decoder 132, which generates predicted billing codes 133. The predicted billing codes are predictions of good billing codes to add to the physician's fee schedule 111. The concatenation, or other combination, of vectors permits the decoder 132 to use information from both the physician's fee schedule and recent billing claims to make its prediction. In some embodiments, the decoder 132 is a neural network. In some embodiments, the decoder 132 is an RNN. In some embodiments, the decoder 132 is an LSTM neural network.

Decoder 132 may output predicted billing codes 133 in an encoded vector representation in the same format and vector space used by the encoded billing codes 103. The decoder 132 may then map the encoded version of the predicted billing codes to a raw format, providing the actual billing codes themselves. This may be accomplished by using a similarity metric, such as cosine similarity, to find the most similar encoded billing code to the predicted vector according to the similarity metric, and the decoder 132 predicting the most similar encoded billing code to the predicted vector as the output.

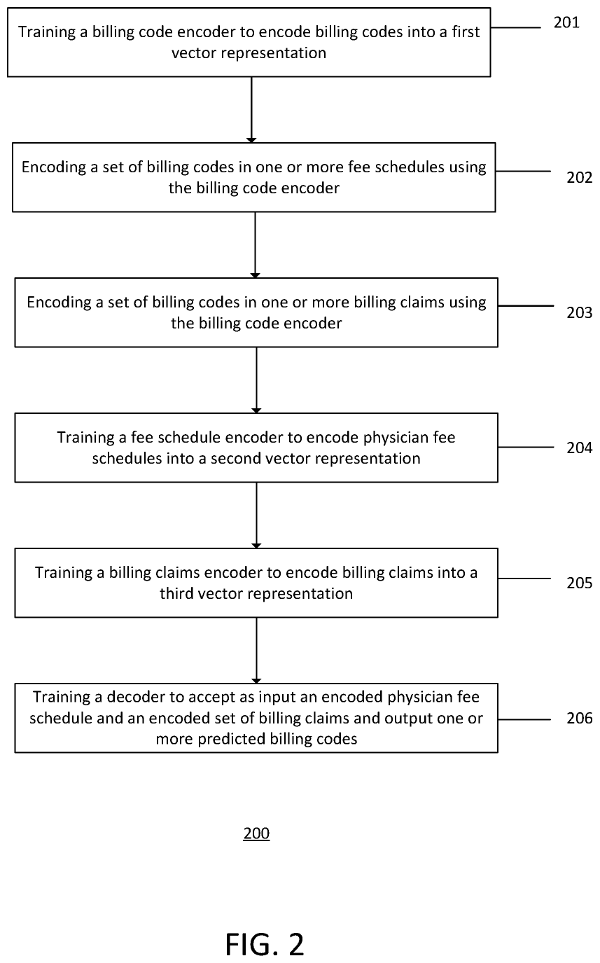

FIG. 2 illustrates an exemplary method 200 for training machine learning system 100 that may be used in an embodiment. The training data for the machine learning system 100 may include one or more ground-truth examples of physician fee schedules and recent billing claims.

In step 201, billing code encoder 102 may be trained to encode billing codes 101 into a first vector representation.

In step 202, a set of billing codes in raw format in a fee schedule may be encoded using the billing code encoder 102 in order to create fee schedule 111 represented with encoded billing codes 115.

In step 203, a set of billing codes in raw format in one or more recent billing claims may be encoded using the billing code encoder 102 in order to create billing claims 121 represented with encoded billing codes 125. The recent billing claims are from the same physician as the fee schedule used in step 202.

In step 204, fee schedule encoder 112 may be trained to encode physician fee schedules in a second vector representation.

In step 205, billing claims encoder 122 may be trained to encode billing claims into a third vector representation.

The first vector representation, second vector representation, and third vector representation may each be distinct representations from each other. The first vector representation is in a vector space of billing codes. The second vector representation is in a vector space of fee schedules. The third vector representation is in a vector space of sets of billing claims.

In step 206, decoder 132 may be trained to accept as input an encoded physician fee schedule and an encoded set of billing claims and produce as output one or more predicted billing codes 133. The decoder 132 may be trained to produce a stop token if no additional billing codes should be added to the physician fee schedule. Backpropagation may be used to train decoder 132 to reduce, or minimize, the error between the actual output of the decoder 132 and the desired output.

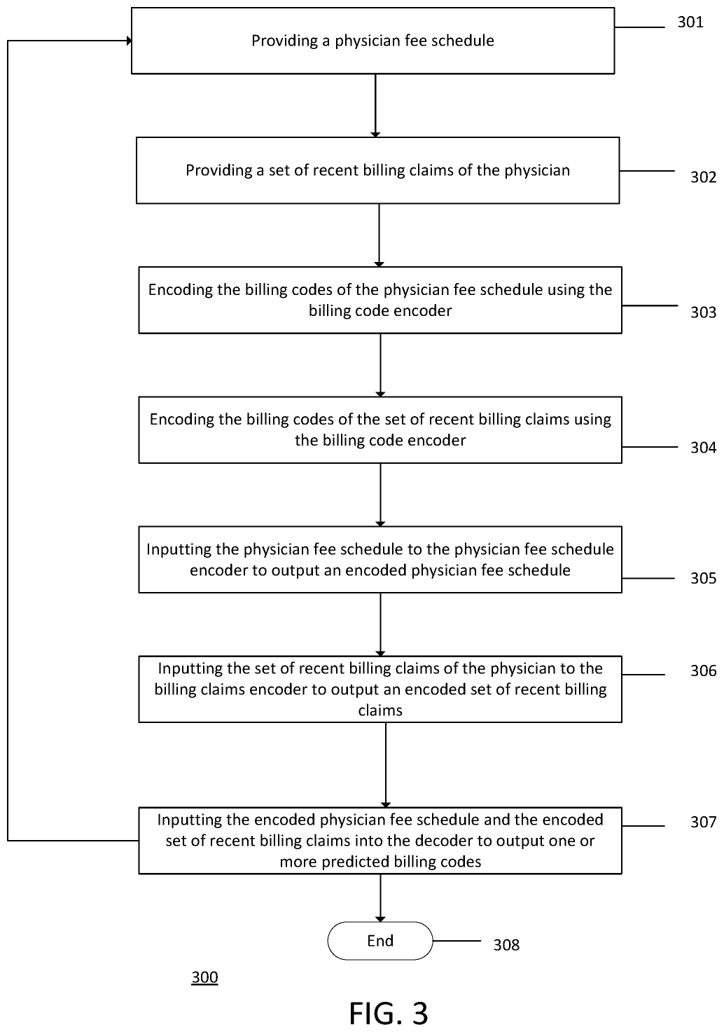

FIG. 3 illustrates an exemplary method 300 that may be used in an embodiment. Method 300 is a method of using machine learning system 100 to produce one or more predicted billing codes for a physician based on the physician's fee schedule and recent set of billing claims.

In step 301, a physician fee schedule may be provided, where the billing codes of the fee schedule may initially be in raw format.

In step 302, a set of recent billing claims of the physician may be provided, where the billing codes of the billing claims may initially be in raw format.

In step 303, the billing codes of the physician fee schedule may be encoded using the billing code encoder 102. This outputs a physician fee schedule comprising a set of encoded billing codes.

In step 304, the billing codes of the set of recent billing claims may be encoded using the billing code encoder 102. This outputs a set of recent billing claims comprising a set of encoded billing codes.

In step 305, the physician fee schedule, comprising a list of encoded billing codes, may be input to the physician fee schedule encoder 112 to output an encoded physician fee schedule.

In step 306, the set of recent billing claims, comprising a list of encoded billing codes, may be input to the billing claims encoder 122 to output an encoded set of recent billing claims of the physician.

In step 307, the encoded physician fee schedule and encoded set of recent billing claims may be combined, such as by concatenation. The combined vector may be input to the decoder 132 to output one or more predicted billing codes or a stop token.

If the decoder 132 outputs the stop token, then the method ends at step 308. However, if the decoder 132 outputs a predicted billing code, then the method may continue by adding the predicted billing code to the physician's fee schedule and then repeating method 300 from step 301 on the new fee schedule including the predicted billing code. The method 300 may repeat iteratively, adding newly predicted billing codes to the physician's fee schedule until a stop token is output by the decoder 132 or a maximum length is reached.

In some embodiments, beam search is used to follow multiple potential paths of adding predicted billing codes. The use of beam search to follow multiple paths aids in avoiding the method getting stuck at a local maximum without reaching a potentially higher maximum elsewhere. Beam search is an algorithm with a configurable branching factor n, which defines the number of paths that are followed.

When beam search is used, in step 307 the decoder 132 outputs a vector representation of a billing code or a stop token. A set of n billing codes are chosen to be added in separate branches of the beam search. In some embodiments, the n closest billing codes to the output vector representation are chosen. However, other methods of selection are also possible, such as adding a small random number to the output vector representation and then choosing the closest billing codes to obtain more diversity of outcomes. Another iteration of method 300 is performed for each of the n most promising branches.

At each iteration, the n most promising additions of predicted billing codes are added to the fee schedule, each in a separate branch of the search, and the beam search continues to iterate. The beam search ends when all branches have terminated with a stop token or have reached a maximum length. When the beam search has ceased, the algorithm outputs the set of billing codes—the predicted fee schedule—having the highest predicted probability.

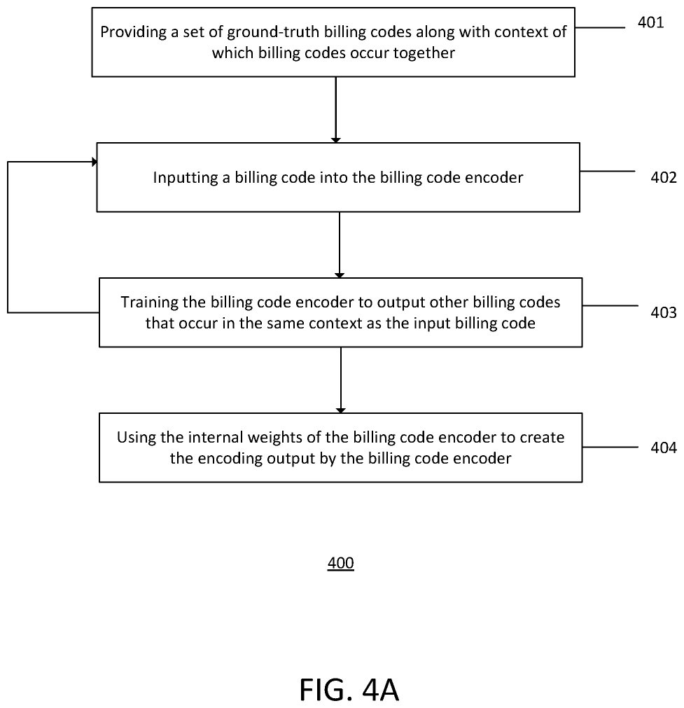

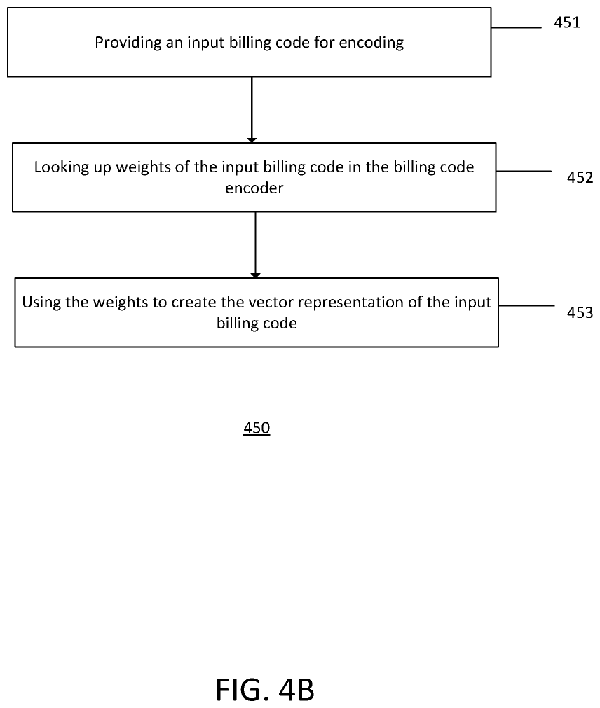

FIG. 4A illustrates an exemplary method 400 for training the billing code encoder 102, which may be used in some embodiments.

In step 401, a set of ground-truth billing codes is provided along with context of which billing codes occur together. For example, the ground-truth billing codes may be provided as a set of millions of billing claims that were submitted by physicians, or that were accepted for payment by a payer. Through method 400, the billing code encoder 102 is trained to output vector representations so that billing codes that commonly occur together are encoded to be closer together in the vector space than billing codes that do not commonly occur together.

In step 402, a billing code is input into the billing code encoder 102. In some embodiments, the billing code encoder 102 uses the skip-gram model. In the skip-gram model, the billing code is input as a one-hot encoding. A one-hot encoding is a vector with all the entries of the vector equal to ‘0’ except for a single ‘1.’ Each entry in the vector represents a single item, and the location of the ‘1’ indicates the item that is represented by the vector. Therefore, a one-hot encoding of billing codes means that the vector has length equal to the number of billing codes and each entry represents a single billing code. In the skip-gram model, the billing code encoder 102 may be a single layer neural network.

In step 403, the billing code encoder 102 is trained to output other billing codes that occur in the same claim as the input billing code. This may be performed by inputting the other billing codes that occur in the claims as a correct output for the input to billing code encoder 102 and performing backpropagation in the neural network comprising the billing code encoder 102. The output billing codes may also be represented by one-hot encodings. Steps 402 and 403 may be repeated for multiple billing codes.

In step 404, in a skip-gram model, the internal weights of the billing code encoder 102 may be used to generate the output encoding of the billing code encoder 102. In some embodiments, the set of internal weights for the input billing code may be used as the vector representation. In some embodiments, the set of internal weights of the input billing code may be pairwise added to the internal weights for the input billing code in the decoding step of the skip-gram neural network to generate the vector representation.

FIG. 4B illustrates an exemplary method 450 for encoding a billing code that may be used in some embodiments.

In step 451, an input billing code in raw format may be provided for encoding. The input billing code may be transformed into a one-hot encoding.

In step 452, the internal weights in the billing code encoder 102 for the input billing code may be retrieved.

In step 453, the weights may be used by the billing code encoder 102 to generate the vector representation of the input billing code. In some embodiments, the set of internal weights for the input billing code may be used as the vector representation. In some embodiments, the set of internal weights of the input billing code may be pairwise added to the internal weights for the input billing code in the decoding step of the skip-gram neural network to generate the vector representation.

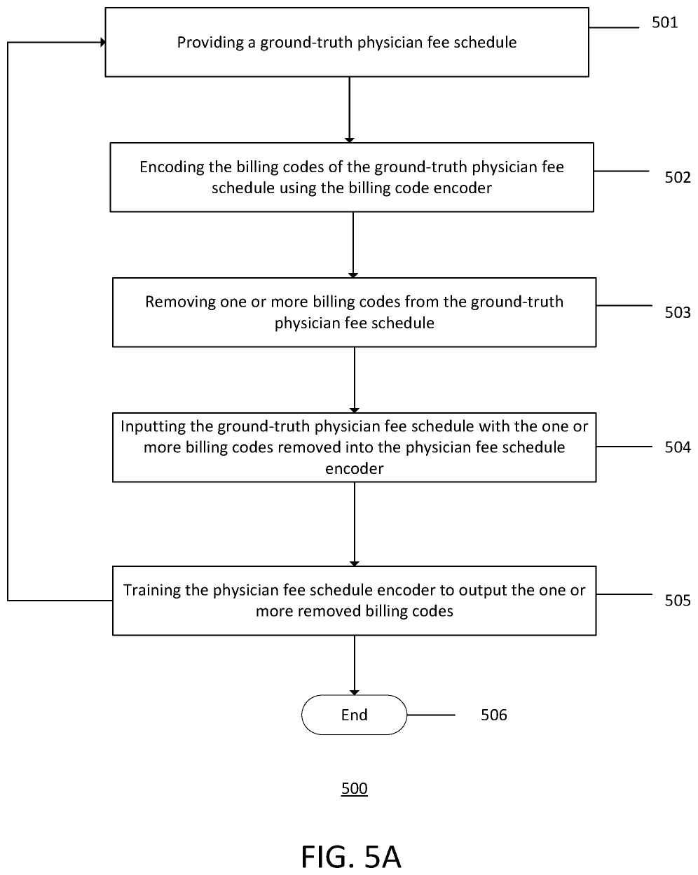

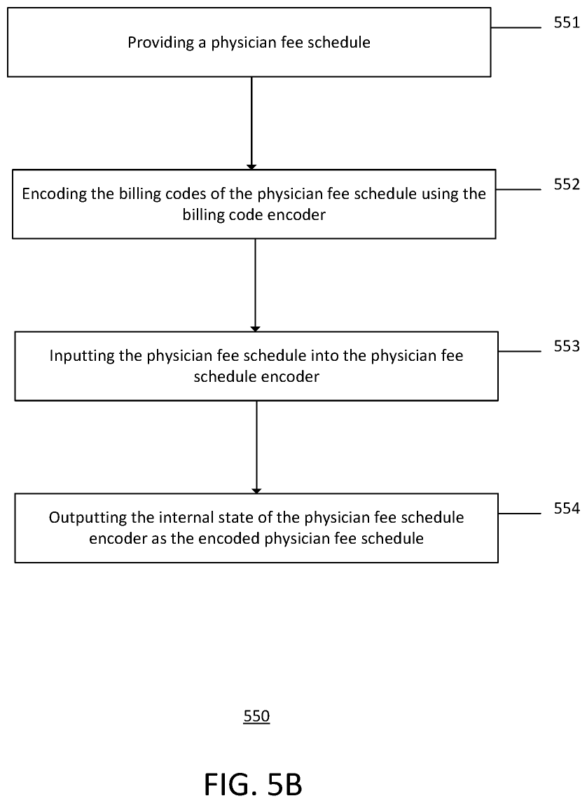

FIG. 5A illustrates an exemplary method 500 for training the fee schedule encoder 112, which may be used in some embodiments.

In step 501, a ground-truth physician fee schedule may be provided. The ground-truth physician fee schedule is a known physician fee schedule that is used for training.

In step 502, the billing codes of the ground-truth physician fee schedule may be encoded using billing code encoder 102 to create encoded billing codes. Optionally, the billing codes of the ground-truth physician fee schedule may be randomly reordered during training to reduce or eliminate the effect of billing code order on the training.

In step 503, one or more billing codes may be removed from the ground-truth physician fee schedule. In this way, the ground-truth physician fee schedule is now missing one or more billing codes that would correctly be included in the ground-truth physician fee schedule.

In step 504, the ground-truth physician fee schedule with the one or more billing codes removed is input into the physician fee schedule encoder 112.

In step 505, the physician fee schedule encoder 112 is trained to output the one or more billing codes that were removed in encoded vector form. This is performed by inputting the encoded versions of the removed billing codes as correct outputs of the physician fee schedule encoder 112 when the encoded ground-truth fee schedule is input in step 504. Backpropagation is used to reduce error between the actual output the physician fee schedule encoder and the encoded versions of the removed billing codes, which are the target output.

The method 500 may repeat at step 501 to train on additional physician fee schedules. The method 500 ends at step 506.

FIG. 5B illustrates an exemplary method 550 of encoding a physician fee schedule that may be used in some embodiments.

In step 551, a physician fee schedule may be provided with the billing codes in raw format.

In step 552, the billing codes of the physician fee schedule may be encoded using the billing code encoder 102 so that each billing code of the physician fee schedule is encoded.

In step 553, the physician fee schedule, comprising encoded billing codes, may be input into the physician fee schedule encoder 112. In some embodiments, the fee schedule encoder 112 is an LSTM, which stores an internal state that is preserved through iterations of the LSTM. In the LSTM, the internal state is stored as a vector and transmitted as input into the next iteration of the LSTM, when a new token (or in this case billing code) is input.

In step 554, the physician fee schedule encoder 112 may output the internal state of the physician fee schedule encoder as the encoded physician fee schedule. In some embodiments, the internal state of the physician fee schedule encoder that is output is the internal state of an LSTM neural network or is based on the internal state of the LSTM neural network.

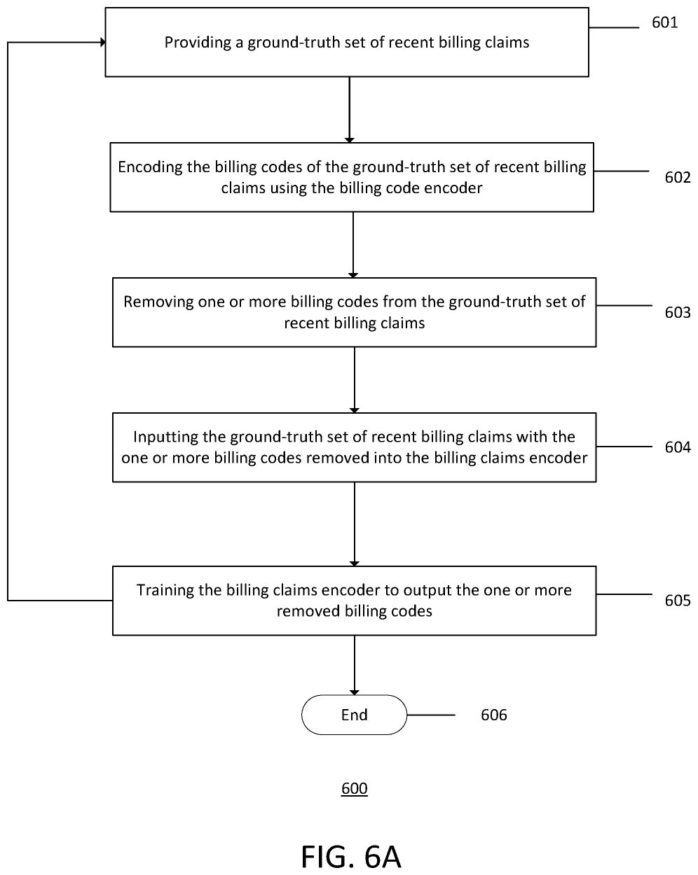

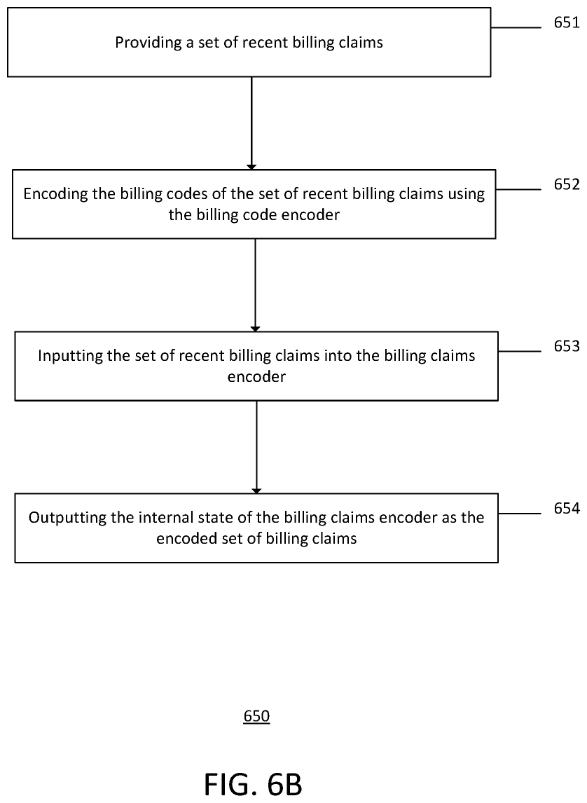

FIG. 6A illustrates an exemplary method 600 for training the billing claims encoder 122, which may be used in some embodiments.

In step 601, a ground-truth set of recent billing claims may be provided. The ground-truth set of recent billing claims is a known set of recent billing claims that is used for training.

In step 602, the billing codes of the ground-truth set of recent billing claims may be encoded using billing code encoder 102 to create encoded billing codes. Optionally, the billing codes of the set of recent billing claims may be randomly reordered during training to reduce or eliminate the effect of billing code order on the training.

In step 603, one or more billing codes may be removed from the ground-truth set of recent billing claims. In this way, the ground-truth set of recent billing claims is now missing one or more billing codes that would correctly be included in the ground-truth set of recent billing claims.

In step 604, the ground-truth set of recent billing claims with the one or more billing codes removed is input into the billing claims encoder 122.

In step 605, the billing claims encoder 122 is trained to output the one or more billing codes that were removed in encoded vector form. This is performed by inputting the encoded versions of the removed billing codes as correct outputs of the billing claims encoder 122 when the encoded ground-truth set of recent billing claims is input in step 604. Backpropagation is used to reduce error between the actual output of the billing claims encoder 122 and the encoded versions of the removed billing codes, which are the target output.

FIG. 6B illustrates an exemplary method 650 of encoding a set of recent billing claims that may be used in some embodiments.

In step 651, a set of recent billing claims may be provided with the billing codes in raw format.

In step 652, the billing codes of the set of recent billing claims may be encoded using the billing code encoder 102 so that each billing code of the physician fee schedule is encoded.

In step 653, the set of recent billing claims, comprising encoded billing codes, may be input into the billing claims encoder 122. In some embodiments, the billing claims encoder 122 is an LSTM, which stores an internal state that is preserved through iterations of the LSTM. In the LSTM, the internal state is stored as a vector and transmitted as input into the next iteration of the LSTM, when a new token (or in this case billing code) is input.

In step 654, the billing claims encoder 122 may output the internal state of the billing claims encoder as the encoded set of billing claims. In some embodiments, the internal state of the billing claims encoder 122 that is output is the internal state of an LSTM neural network or is based on the internal state of the LSTM neural network.

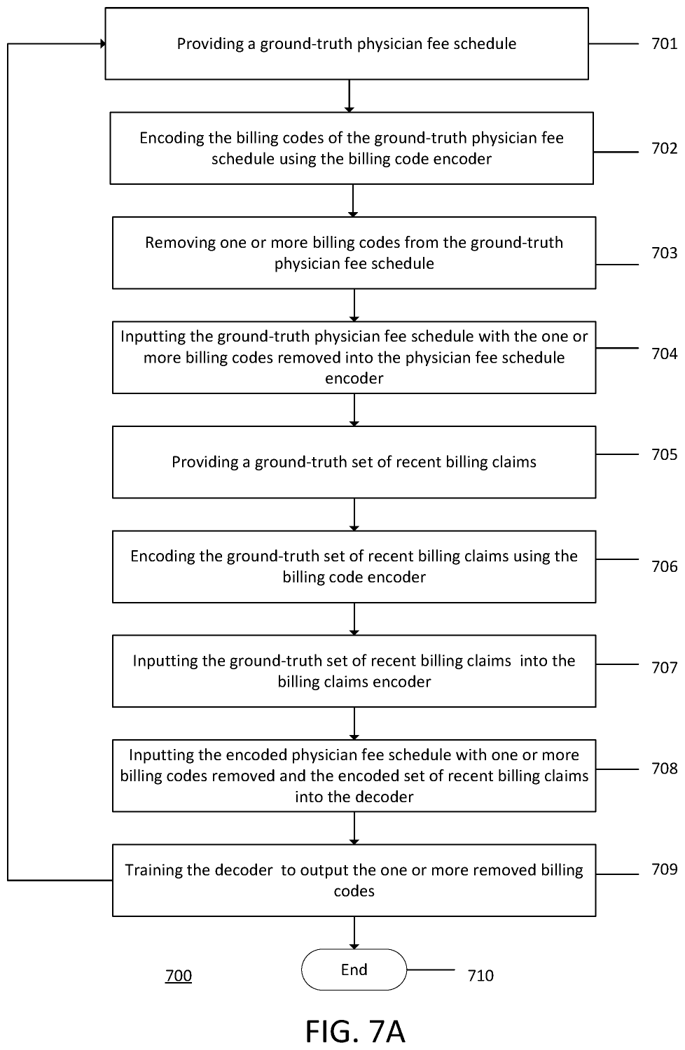

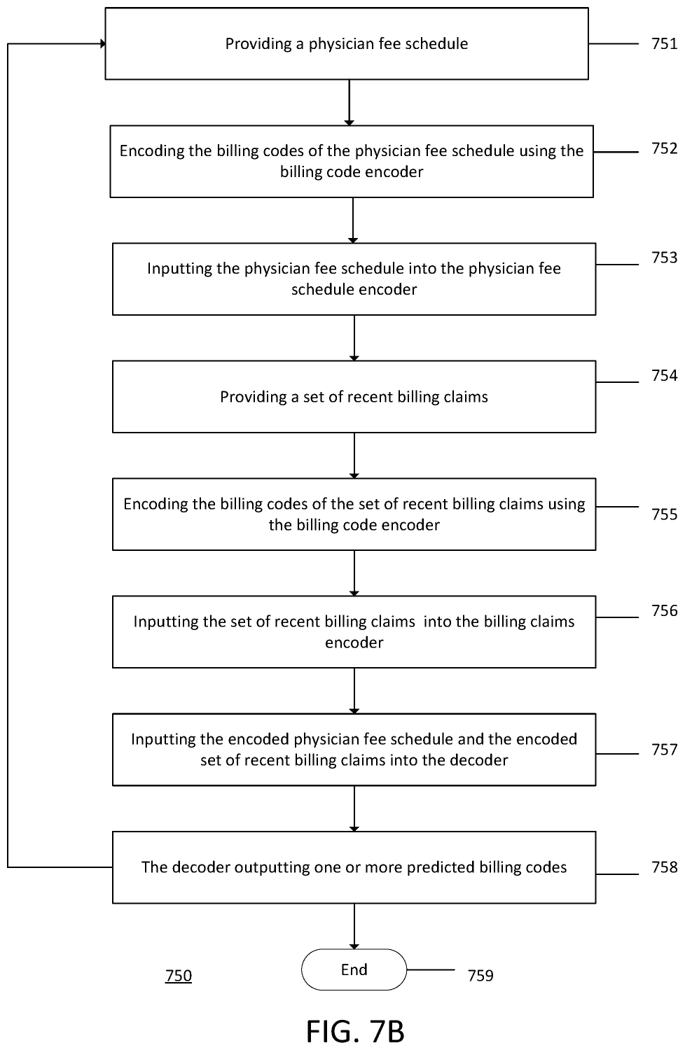

FIG. 7A illustrates an exemplary method 700 of training the decoder 132 to correctly predict billing codes to add to a fee schedule, which may be used in some embodiments.

In step 701, a ground-truth physician fee schedule may be provided.

In step 702, the billing codes of the ground-truth physician fee schedule may be encoded using the billing code encoder 102. This produces a physician fee schedule comprising encoded billing codes.

In step 703, one or more billing codes are removed from the ground-truth physician fee schedule.

In step 704, the ground-truth physician fee schedule with the one or more billing codes removed is input into the physician fee schedule encoder 112 to output an encoded physician fee schedule with the one or more billing codes removed.

In step 705, a ground-truth set of recent billing claims of the physician may be provided, for the physician corresponding to the provided fee schedule.

In step 706, the billing codes of the ground-truth set of recent billing claims may be encoded using the billing code encoder 102. This produces a set of recent billing claims comprising encoded billing codes.

In step 707, the ground-truth set of recent billing claims is input into the billing claims encoder 122 to output an encoded set of recent billing claims of the physician.

In step 708, the encoded physician fee schedule with the one or more billing claims removed and the encoded set of recent billing claims may be combined, such as by concatenation. The resulting vector may be input into the decoder 132.

In step 709, the decoder 132 is trained to output one or more removed billing codes. This is performed by inputting the encoded versions of the removed billing codes as correct outputs of the decoder 132 when the encoded physician fee schedule with the one or more billing claims removed and the encoded set of recent billing claims are input in step 708. Backpropagation is used to reduce error between the actual output of the decoder 132 and the encoded versions of the removed billing codes, which are the target output. The process may be repeated to train with additional fee schedules.

FIG. 7B illustrates an exemplary method 750 of using decoder 132, after decoder 132 has been trained, to output predicted billing codes for a fee schedule according to some embodiments.

In step 751, a physician fee schedule is provided.

In step 752, the billing codes of the physician fee schedule are encoded using the billing code encoder 102.

In step 753, the physician fee schedule, comprising encoded billing codes, is input into the physician fee schedule encoder 112 to output an encoded physician fee schedule.

In step 754, a set of recent billing claims is provided, the recent billing claims corresponding to the same physician for whom the physician fee schedule was provided.

In step 755, the billing codes of the set of recent billing claims are encoded using the billing code encoder 102.

In step 756, the set of recent billing claims, comprising encoded billing codes, is input into the billing claims encoder 122 to output an encoded set of recent billing claims.

In step 757, the encoded physician fee schedule and the encoded set of recent billing claims are combined, such as by concatenation. The resulting vector is input into the decoder 132.

In step 758, the decoder 132 outputs vector representations of one or more billing codes and maps the vector representations to the original format of the billing codes by finding the most similar billing code in the vector space.

The predicted billing code may be added to the fee schedule and the process repeated. However, if decoder 132 outputs a stop token or reaches a maximum length of billing codes in the fee schedule, then the process ends at step 759. Beam search may be used to follow multiple possible paths and avoid being stuck in a local maximum.

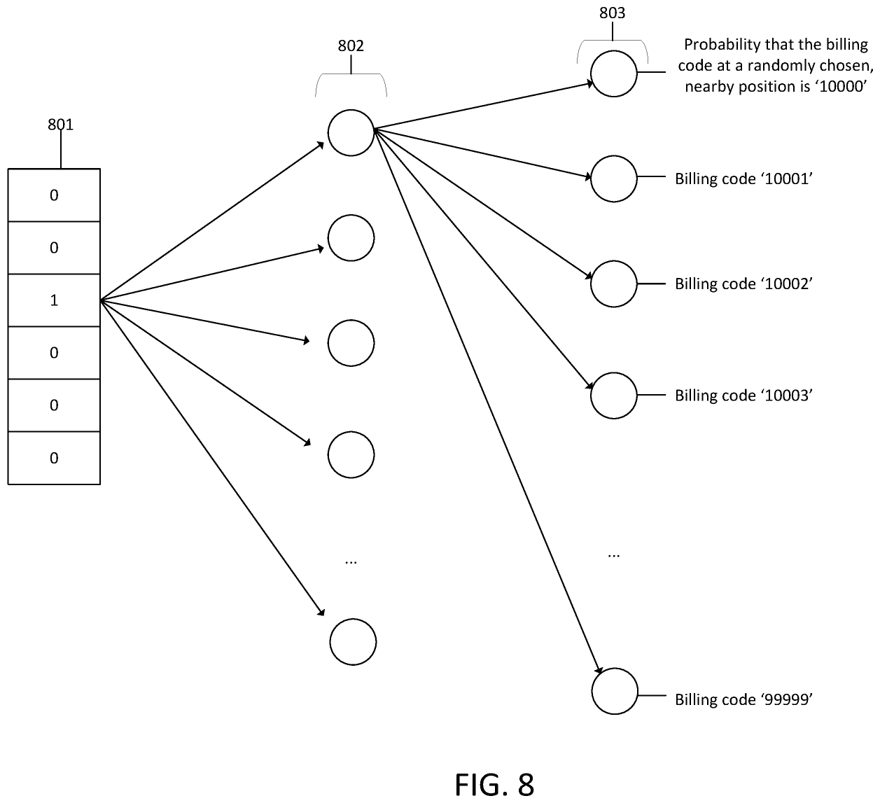

FIG. 8 illustrates a skip-gram model, which may be used in some embodiments to implement billing code encoder 102. The skip-gram model is a one layer neural network trained to accept one-hot encoding of billing codes as inputs and output a probability distribution of the likely billing codes to appear in the same claim. A one-hot encoding of a billing code 801 is accepted as the input to a single hidden layer of neurons 802. The single hidden layer is connected to an output layer of neurons 803. Each layer is fully-connected. The hidden layer of neurons 802 may use a linear activation function for their output, and the output layer of neurons 803 may use the Softmax function as the activation function. After training the skip-gram model on ground-truth examples of billing codes and other billing codes appearing in the same billing claim, the weights in the hidden layer 802 for each one-hot encoding may be used as the vector representation for the corresponding billing code of the one-hot encoding.

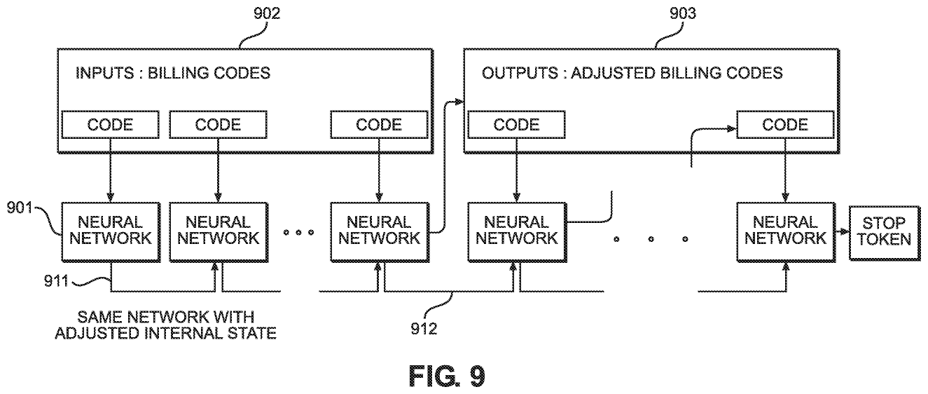

FIG. 9 illustrates an LSTM neural network 901, which may be used to implement the fee schedule encoder 112, billing claims encoder 122, and decoder 132. The LSTM neural network 901 comprises a plurality of neural network nodes. The neural network nodes may have an activation function that defines the output of the neural network nodes. Some of the neural network nodes may have a sigmoid activation function and other of the neural network nodes may have a tanh (hyperbolic tangent function) activation function. The LSTM neural network 901 may also have one or more gates to control the flow of information. Some of the gates may perform pointwise addition, and other of the gates may perform pointwise multiplication.

When used to implement the fee schedule encoder 112 and billing claims encoder 122, the LSTM neural network accepts input 902 comprising a sequence of billing codes. As described above, the billing codes may be encoded using the billing code encoder 102. The billing codes 902 are input sequentially to the LSTM neural network 901. At each step, the LSTM neural network 901 takes as input the next billing code from billing codes 902, and an internal state passed from the prior iteration, such as state 911 passed from the first iteration to the second iteration in the diagram. The internal state may be represented as a vector. At each iteration, the LSTM neural network 901 outputs internal state and also an output representing a billing code, in an encoded vector representation, or a stop token.

The output billing codes are ignored until the last code of the input 902 has been input to the LSTM neural network 901. At that point, the billing code output by the LSTM neural network 901 becomes part of the output 903, which may be predicted billing codes to be added to, for example, the fee schedule. The output billing codes 903 may be added to the physician fee schedule and process continued to predict additional output billing codes 903 that may also be added to the physician fee schedule. The process may end when a stop token or a maximum length is reached.

During the encoding process, the internal state vector 912 output by the LSTM neural network 901 after the last input billing code 902 has been input may be the vector representation used for the encoded fee schedule, when using the fee schedule encoder 112, and the encoded set of billing claims, when using the billing claims encoder 122.

The terminology used herein is for the purpose of describing particular aspects only and is not intended to be limiting of the disclosure. As used herein, the singular forms “a,” “an,” and “the” are intended to comprise the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

While the invention has been particularly shown and described with reference to specific embodiments thereof, it should be understood that changes in the form and details of the disclosed embodiments may be made without departing from the scope of the invention. Although various advantages, aspects, and objects of the present invention have been discussed herein with reference to various embodiments, it will be understood that the scope of the invention should not be limited by reference to such advantages, aspects, and objects. Rather, the scope of the invention should be determined with reference to patent claims.

What is claimed:

1. A computer-implemented method for recommending one or more codes for a physician code schedule, the method comprising:
training a first neural network encoder to encode codes into a first vector representation, the first vector representation relating codes that are similar;
training a second neural network encoder to encode physician code schedules into a second vector representation, the second vector representation relating physician code schedules that are similar, wherein the physician code schedules comprise one or more codes;
training a third neural network encoder to encode claims into a third vector representation, the third vector representation relating claims that are similar, wherein the claims comprise one or more codes;
training a neural network decoder to accept as input an encoded physician code schedule and an encoded set of claims to output one or more predicted codes;
recommending a code to add to a physician code schedule of a physician by:
providing the physician code schedule;
providing a set of recent claims of the physician;
inputting the physician code schedule to the second neural network encoder to output an encoded physician code schedule;
inputting the set of recent claims of the physician to the third neural network encoder to output an encoded set of recent claims of the physician;
inputting the encoded physician code schedule and the encoded set of recent claims of the physician into the neural network decoder to output a predicted code.

2. The computer-implemented method of claim 1, wherein the first neural network encoder creates word embeddings of codes by using a skip-gram model.

3. The computer-implemented method of claim 1, wherein the second neural network encoder is a long short-term memory (LSTM) neural network, and
the second vector representation is created based on the internal state of the second neural network encoder.

4. The computer-implemented method of claim 1, wherein the second neural network encoder is trained by:
removing one or more codes from a ground-truth physician code schedule;
inputting the ground-truth physician code schedule with the one or more codes removed into the second neural network encoder; and
training the second neural network encoder to output the one or more removed codes.

5. The computer-implemented method of claim 1, wherein the third neural network encoder is a long short-term memory (LSTM) neural network, and
the third vector representation is created based on the internal state of the third neural network encoder.

6. The computer-implemented method of claim 1, wherein the third neural network encoder is trained by:
removing one or more codes from a ground-truth set of recent claims;
inputting the ground-truth set of recent claims with the one or more codes removed into the third neural network encoder; and
training the third neural network encoder to output the one or more removed codes.

7. The computer-implemented method of claim 1, wherein the neural network decoder is trained by:
removing one or more codes from a ground-truth physician code schedule;
inputting the ground-truth physician code schedule with the one or more codes removed into the second neural network encoder to output an encoded ground-truth physician code schedule with the one or more codes removed;
providing a ground-truth set of recent claims;
inputting the ground-truth set of recent claims into the third neural network encoder to output an encoded ground-truth set of recent claims;
inputting the encoded ground-truth physician code schedule with the one or more codes removed and the encoded ground-truth set of recent claims in the neural network decoder, and
training the neural network decoder to output the one or more removed codes.

8. A computer-implemented method for recommending one or more codes for a physician code schedule, the method comprising:
training a first encoder to encode codes into a first vector representation, the first vector representation relating codes that are similar;
training a second encoder to encode physician code schedules into a second vector representation, the second vector representation relating physician code schedules that are similar, wherein the physician code schedules comprise one or more codes;
training a third encoder to encode claims into a third vector representation, the third vector representation relating claims that are similar, wherein the claims comprise one or more codes;
training a decoder to accept as input an encoded physician code schedule and an encoded set of claims to output one or more predicted codes;
recommending a code to add to a physician code schedule of a physician by:
providing the physician code schedule;
providing a set of recent claims of the physician;
inputting the physician code schedule to the second encoder to obtain an encoded physician code schedule;
inputting the set of recent claims of the physician to the third encoder to obtain an encoded set of recent claims of the physician;
inputting the encoded physician code schedule and the encoded set of recent claims of the physician into the decoder to output a predicted code.

9. The computer-implemented method of claim 8, wherein the first encoder creates word embeddings of codes by using a skip-gram model.

10. The computer-implemented method of claim 8, wherein the second encoder is a long short-term memory (LSTM) neural network, and
the second vector representation is created based on the internal state of the second neural network encoder.

11. The computer-implemented method of claim 8, wherein the second encoder is trained by:
removing one or more codes from a ground-truth physician code schedule;
inputting the ground-truth physician code schedule with the one or more codes removed into the second encoder; and
training the second encoder to output the one or more removed codes.

12. The computer-implemented method of claim 8, wherein the third encoder is a long short-term memory (LSTM) neural network, and
the third vector representation is created based on the internal state of the third encoder.

13. The computer-implemented method of claim 8, wherein the third encoder is trained by:
removing one or more codes from a ground-truth set of recent claims;
inputting the ground-truth set of recent claims with the one or more codes removed into the third encoder; and
training the third encoder to output the one or more removed codes.

14. The computer-implemented method of claim 8, wherein the decoder is trained by:
removing one or more codes from a ground-truth physician code schedule;
inputting the ground-truth physician code schedule with the one or more codes removed into the second encoder to output an encoded ground-truth physician code schedule with the one or more codes removed;
providing a ground-truth set of recent claims;
inputting the ground-truth set of recent claims into the third encoder to output an encoded ground-truth set of recent claims;
inputting the encoded ground-truth physician code schedule with the one or more codes removed and the encoded ground-truth set of recent claims in the decoder, and
training the decoder to output the one or more removed codes.

15. A computer-implemented method for recommending one or more codes for a physician code schedule, the method comprising:
training a first encoder to encode codes into a first vector representation;
training a second encoder to encode physician code schedules into a second vector representation;
training a decoder to accept as input an encoded physician code schedule to output one or more predicted codes;
recommending a code to add to a physician code schedule of a physician by:
providing the physician code schedule;
inputting the physician code schedule to the second encoder to obtain an encoded physician code schedule;
inputting the encoded physician code schedule into the decoder to output a predicted code.

16. The computer-implemented method of claim 15, wherein the first encoder creates word embeddings of codes by using a skip-gram model.

17. The computer-implemented method of claim 15, wherein the second encoder is a long short-term memory neural network (LSTM), and
the second vector representation is created based on the internal state of the second neural network encoder.

18. The computer-implemented method of claim 15, wherein the second encoder is trained by:
removing one or more codes from a ground-truth physician code schedule;
inputting the ground-truth physician code schedule with the one or more codes removed into the second encoder; and
training the second encoder to output the one or more removed codes.

19. The computer-implemented method of claim 15, wherein the decoder is trained by:
removing one or more codes from a ground-truth physician code schedule;
inputting the ground-truth physician code schedule with the one or more codes removed into the second encoder to output an encoded ground-truth physician code schedule with the one or more codes removed;
inputting the encoded ground-truth physician code schedule with the one or more codes removed in the decoder, and
training the decoder to output the one or more removed codes.

20. The computer-implemented method of claim 15, wherein the second encoder is a neural network comprising at least one node having a sigmoid activation function and at least one node having a tanh activation function.
